(19) United States
(12) Patent Application Publication
(10) Pub. No.: US 2009/0270795 A1
(43) Pub. Date: Oct. 29, 2009

(54) METHOD FOR WELDING A BARRIER IN AN IMPLANTABLE DRUG DELIVERY DEVICE

(75) Inventors: Ahmed Elmouelhi, Minneapolis, MN (US); Thomas W. Hovind, Andover, MN (US); Chris J. Paidosh, St. Anthony, MN (US)

Correspondence Address: SHUMAKER & SIEFFERT , P.A, 1625 RADIO DRIVE , SUITE 300, WOODBURY, MN, 55125, US

(73) Assignee: Medtronic, Inc., Minneapolis (US)

(21) Appl. No.: 12/108,242

(22) Filed: Apr. 23, 2008

(51) Int. Cl. A61M 1/00; B21D 39/03; B23K 9/28

(52) U.S. Cl. 604/27; 29/428; 219/86.1

(57) ABSTRACT

A method including laser welding a barrier to a sleeve and at least one of a housing or coil cup to enclose a solenoid in a solenoid-driven piston pump. The solenoid driven piston pump may comprise a portion of an implantable drug delivery device.

### TECHNICAL FIELD

The invention relates to implantable drug delivery devices, and more specifically implantable drug delivery devices including a solenoid driven piston pump.

### BACKGROUND

An implantable drug delivery device can be implanted by a clinician into a patient at a location appropriate for the therapy that interferes as little as practicable with patient activity, such as subcutaneously in the lower abdomen. Typically, a drug delivery catheter is connected to a drug pump outlet and implanted to infuse the drug, infusate or other therapeutic substance at a programmed infusion rate and predetermined location to treat a medical condition. Reliable and accurate operation of the drug pump is important because both inadequate and unintended therapeutic substance delivery can create patient complications. Many drug pumps are configured so the pump can be replenished with drug through a refill port or septum while the pump is implanted, so the period of time in which the pump is implanted is not limited by drug capacity. In electrically powered implantable drug pumps, the period the pump can be implanted can be limited by factors such as battery consumption, corrosive damage, and mechanical wear. The relatively large size of some implantable drug pumps can limit locations where the device can be implanted in a patient.

### SUMMARY

In general, the disclosure is directed to a method for welding a barrier to a housing as a part of a solenoid-driven piston pump and an implantable drug delivery device including features that facilitate the welding of the barrier. In some embodiments, the barrier may include a titanium foil of a thickness of less than about 0.004 inches, about 0.002 inches, or less than about 0.002 inches.

In one aspect, the disclosure is directed to a method including providing an implantable drug delivery device including a housing defining a depression. The housing comprises a first surface. The method further includes inserting a bore housing including a coil cup and a sleeve in the depression. The coil cup includes a coil cup base plate having a first major surface and a second major surface opposite the first major surface. The coil cup base plate defines a coil cup perimeter and a coil cup orifice located substantially centered in the coil cup base plate. The coil cup also includes a coil cup perimeter wall including a perimeter wall top surface, attached to the first major surface of the coil cup base plate along the coil cup perimeter, and projecting substantially perpendicular to the first major surface of the coil cup base plate. The coil cup further includes a coil cup inner wall attached to the first major surface of the coil cup base plate along a perimeter of the coil cup orifice and projecting substantially perpendicular to the first major surface of the coil cup base plate. The coil cup inner wall further defines the coil cup orifice, and an outer surface of the coil cup inner wall, an inner surface of the coil cup perimeter wall and the first major surface of the coil cup base plate define a bore housing cavity. The sleeve includes a sleeve top surface and a sleeve inner surface. The sleeve is press fit into the coil cup orifice and defines a bore housing orifice. When the bore housing is inserted in the depression, the first surface of the housing, the perimeter wall top surface and the sleeve top surface are substantially co-planar. The method also includes placing a solenoid in the bore housing cavity, placing a pin in the bore housing orifice, and placing a barrier over the bore housing. The barrier defines a barrier perimeter and a barrier orifice substantially centered in the barrier. The pin substantially centers the barrier orifice over the bore housing orifice and the barrier substantially covers the bore housing cavity. The method further includes clamping the barrier against the bore housing with a clamping structure and spot welding the barrier perimeter to at least one of the first surface of the housing and the perimeter wall top surface at a plurality of spot weld locations with a laser. The method also includes removing the clamping structure, removing the pin, welding a remaining section of the barrier perimeter to the first surface of the housing with the laser, and welding a perimeter of the barrier orifice to the sleeve top surface with the laser.

In another aspect, the disclosure is directed to an implantable drug delivery device including a housing. The housing includes a depression and a first surface. The drug delivery device also includes a bore housing inserted in the depression, and the bore housing includes a coil cup and a sleeve. The coil cup includes a coil cup base plate having a first major surface and a second major surface opposite the first major surface. The coil cup base plate defines a coil cup perimeter and a coil cup orifice located substantially centered in the coil cup base plate. The coil cup also includes a coil cup perimeter wall that includes a perimeter wall top surface, attached to the first major surface of the coil cup base plate along the coil cup perimeter and projecting substantially perpendicular to the first major surface of the coil cup base plate. The coil cup further comprises a coil cup inner wall attached to the first major surface of the coil cup base plate along a perimeter of the coil cup orifice and projecting substantially perpendicular to the first major surface of the coil cup base plate. The coil cup inner wall further defines the coil cup orifice, and an outer surface of the coil cup inner wall, an inner surface of the coil cup perimeter wall and the first major surface of the coil cup base plate define a bore housing cavity. The sleeve includes a sleeve top surface and a sleeve inner surface. The sleeve is press fit into the coil cup orifice and the sleeve inner surface defines a bore housing orifice. The first surface of the housing, the perimeter wall top surface and the sleeve top surface are substantially co-planar.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A and 1B are block diagrams illustrating cross-sectional views of a piston pump with the piston in a lowered and a raised position, respectively.

FIG. 2 is an exploded perspective view of a bore housing and barrier.

FIGS. 3A-3C are block diagrams illustrating a portion of an implantable drug delivery device including a barrier that is butt welded to a bore housing and a drug device housing.

FIGS. 4A and 4B are block diagrams illustrating a portion of an implantable drug device including a barrier that is lap welded to a bore housing and a drug device housing.

FIG. 5 is a flowchart illustrating an exemplary method for welding a barrier to a bore housing and a drug device housing.

FIGS. 6A-6H are block diagrams illustrating the steps of a method for welding a barrier to a bore housing and a drug device housing.

FIG. 7 is a schematic diagram of a welding process.

### DETAILED DESCRIPTION

As discussed above, many implantable drug delivery devices are relatively large and must be implanted in locations such as the abdomen, which may be undesirable in some embodiments. For example, implanting an implantable drug delivery device in a location remote from the therapy site may require the use of a relatively long catheter. This may increase the strain on the catheter, which may lead to premature failure of the catheter and require more frequent replacement than is desired. For example, implanting the drug delivery device in an abdomen of a patient and routing the catheter to a location along the patient's spinal cord proximate the patient's neck may require the use of a long catheter, and may lead to significant strain on, and even premature failure of, the catheter due to movement of the patient.

In order to enable the implantable drug delivery device to be implanted in a wider range of locations within a patient, it may be desirable to reduce the size of the implantable drug pump. Existing drug delivery devices typically use a peristaltic pump to pump the drug at a prescribed rate. Peristaltic pumps typically operate by a battery-powered electric motor that drives peristaltic rollers over a flexible tube having one end coupled to a therapeutic substance reservoir and the other end coupled to an infusion outlet to pump the therapeutic substance from the therapeutic substance reservoir through the infusion outlet. In an implantable therapeutic substance delivery device including a peristaltic pump, the peristaltic pump is typically one of the largest components in the device and can consume 90% or more of the available battery power. Additionally, the flexible tube used in a peristaltic pump is typically permeable to some therapeutic substance components such as water that can then infiltrate into the hermetically sealed housing and cause corrosion. Further, the flexible tube can expand resulting in decreased accuracy.

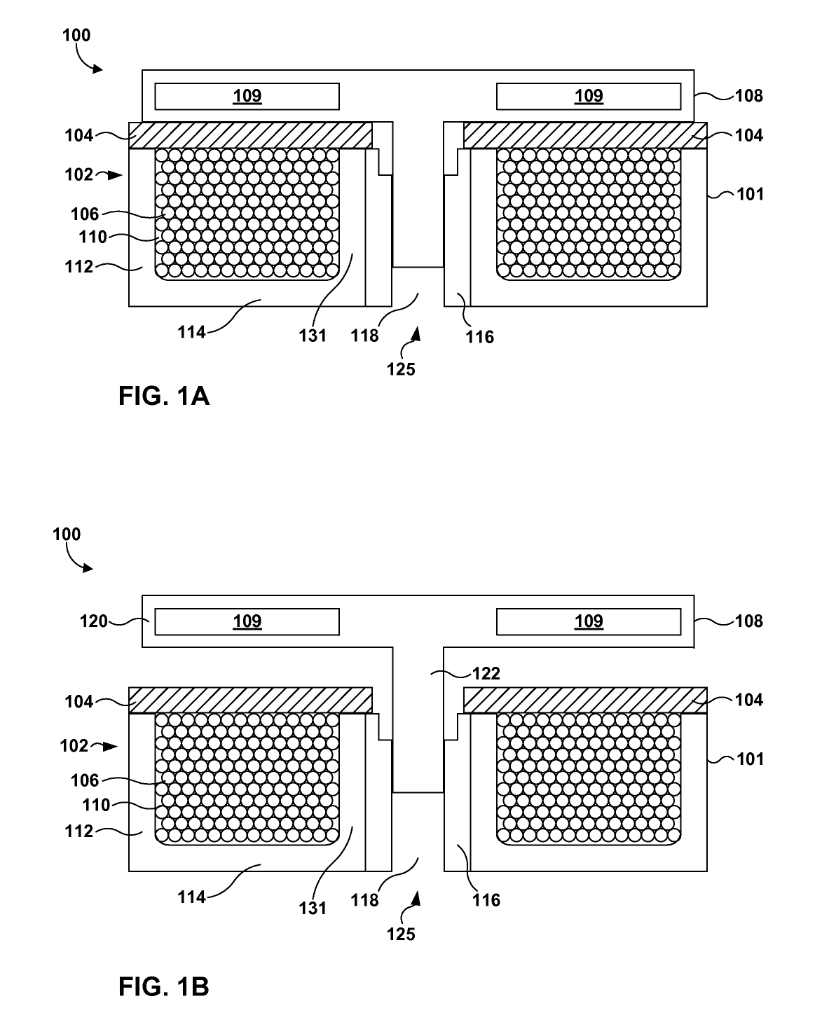

As implantable drug delivery devices are made smaller, the peristaltic pump may be larger than desirable for the housing of the implantable drug delivery device or may consume too much energy, and a new type of pump may be desired or necessary. One alternative pump for an implantable drug delivery device includes a solenoid-driven piston pump. The piston pump may comprise a piston that includes a permanent magnet, and a solenoid that produces a magnetic field that induces motion in the piston. FIGS. 1A and 1B are block diagrams illustrating cross-sections of a piston pump 100 including a solenoid 106. FIG. 1A shows the piston 108 in a lowered position, and FIG. 1B shows the piston 108 in a raised position. The solenoid 106 is located in a bore housing 102, which may include a coil cup 101 and a sleeve 116. The coil cup 101 may include a coil cup base plate 114 and a coil cup inner wall 131 that define a coil cup orifice 125 into which sleeve 116 is press-fit. Sleeve 116 may define a bore housing orifice 118. The coil cup inner wall 131, coil cup perimeter wall 112 and coil cup base plate 114 may define a bore housing cavity 110, in which solenoid 106 is located. Barrier 104 may cover the bore housing cavity 110.

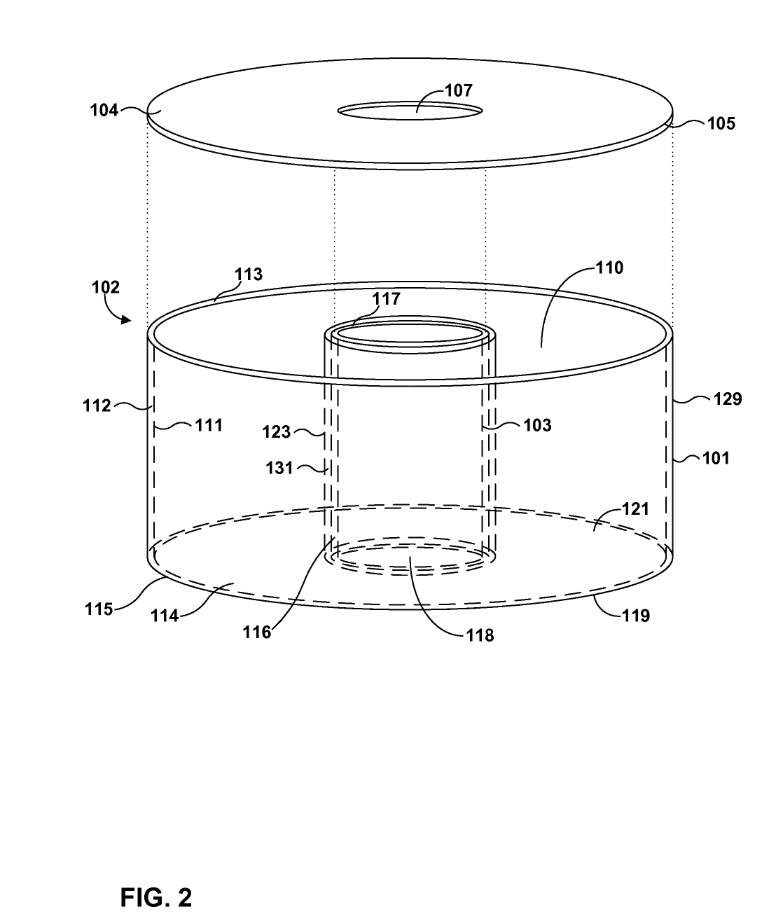

FIG. 2 is an exploded perspective view of bore housing 102 and barrier 104. As seen in FIG. 2, coil cup base plate 114 may include a first major surface 121 and a second major surface 119. Coil cup base plate 114 defines a coil cup perimeter 115 and a coil cup orifice 118 located substantially centered in the base plate 114. Coil cup perimeter wall 112 may be attached to first major surface 121 along coil cup perimeter 115 and may project substantially perpendicular to first major surface 121. Coil cup inner wall 131 may be attached to first major surface along a perimeter of coil cup orifice 125, and further defines coil cup orifice 125, into which sleeve 116 may be press-fit. Sleeve inner surface 103 defines bore housing orifice 118, and inner surface 111 of coil cup perimeter wall 112, coil cup inner wall outer surface 123 and first major surface 121 of coil cup base plate 114 define bore housing cavity 110.

While FIG. 2 illustrates the outer geometry of bore housing 102 as being generally cylindrical, in other embodiments, bore housing 102 may define other outer geometries. For example, coil cup base plate 114 and outer surface 129 of coil cup perimeter wall 112 may generally define a cube. Other outer geometries of bore housing 102 may also be useful in some embodiments. Regardless of the outer geometry of bore housing 102, in some embodiments inner surface 111 of coil cup perimeter wall 112 may be generally cylindrical, as shown in FIG. 2. In other embodiments, inner surface 111 of coil cup perimeter wall 112 may define an alternative geometry, such as that corresponding to the sides of a cube.

Coil cup perimeter wall 112 and sleeve 116 may include a perimeter wall top surface 113 and a sleeve top surface 117, respectively. In some embodiments, perimeter wall top surface 113 and sleeve top surface 117 may be substantially co-planar and provide support for barrier 104.

Barrier 104 defines a barrier perimeter 105 and a barrier orifice 107, which may be substantially centered in barrier 104. That is, a perimeter of barrier orifice 107 may be substantially concentric with barrier perimeter 105. In other embodiments, barrier perimeter 105 may define another geometry, such as a square or rectangle, for example. Barrier orifice 107 may define the same geometry as barrier perimeter 105, or may define a different geometry than barrier perimeter 105. Barrier orifice 107 may substantially align with bore housing orifice 118 when barrier 104 is placed on perimeter wall top surface 113 and sleeve top surface 117.

Referring again to FIGS. 1A and 1B, piston 108 may include a disk-shaped portion 120 and a cylindrical portion 122. Cylindrical portion 122 is disposed in bore housing orifice 118 and slidably engaged with sleeve 116. Disk-shaped portion 120 may include a permanent magnet 109 including poles which are oriented substantially axially in either an N-S or an S-N orientation. Permanent magnet 109 may include a disk-shaped magnet, or may include a number of distinct magnets arranged throughout barrier 104 and magnetically oriented in substantially the same direction.

When a current is passed through the solenoid 106, a magnetic field is generated, which exerts a force on the permanent magnet 109. The direction of the flow of current in the solenoid 106 determines the direction of the magnetic field, and thus the direction of the force applied to the permanent magnet 109. Thus, by changing the direction of current flow, the piston 108 may be made to move from the raised position to the lowered position and vice versa.

Although not illustrated in FIGS. 1A and 1B, the piston pump 100 may further include one or more fluid inlets and one or more fluid outlets, each of which include one-way valves. When the piston 108 moves from the lowered position to the raised position, a negative pressure develops in bore housing orifice 118, which may cause the inlet valve to open and fluid (e.g., the drug) to enter orifice 118. Then, when the piston 108 moves from the raised position to the lowered position, a positive pressure develops in bore housing orifice 118, which closes the inlet valve and opens the outlet valve, pushing the fluid out the outlet valve. A flow rate of the fluid may be determined by the volume change of the orifice during movement of the piston 108 and the number of cycles (e.g., from the lowered position to the raised position and back to the lowered position) of the piston 108 per unit time.

As is well known, a magnetic field at any given point is inversely proportional to distance squared (e.g., B□1/r², where B is the magnetic field and r is distance). In this case, the magnitude of the magnetic field produced by the solenoid 106 at the permanent magnet 109 is proportional to the square of the distance form the solenoid 106 to the permanent magnet 109. Accordingly, it may be desirable to minimize the distance that separates the permanent magnet 109 from the solenoid 106 to reduce the strength of the magnetic field that is necessary to move the piston 108 a desired amount or at a desired rate. For a solenoid 106, the magnetic field is also linearly proportional to the current passing through the solenoid 106 (e.g., B□I, where B is the magnetic field and I is the current). Thus, reducing the necessary magnetic field reduces the necessary current. This is desirable for an implantable drug delivery device, as a battery typically powers the device. Any reduction in current may improve battery life, reduce battery power (and size), or both.

In order to minimize the distance between permanent magnet 109 and solenoid 106, it may be desirable to decrease the thickness of barrier 104. For example, each reduction of thickness of barrier 104 to half the original thickness of barrier 104 may reduce the necessary magnetic field by a factor of four. Along with the physical thickness of barrier 104, any non-planarity in barrier 104 may increase the distance between permanent magnet 109 and solenoid 106. Barrier 104 may be more likely to deviate from planarity as the thickness of barrier 104 is reduced, because the stiffness of the barrier 104 is reduced as the barrier is made thinner. Thus, manufacturing a thin barrier 104 (e.g., about 0.002 in. or less), such as stamping barrier 104 from a sheet of metal, can result in a barrier 104 that deviates from planarity.

While it may be desirable to minimize the thickness of the barrier 104 to improve the efficiency of the pump 100, it is also important to maintain a hermetic seal of the cavity 110. The materials used in constructing the solenoid 106 are typically not biocompatible, and may corrode in the presence of bodily fluids or the therapeutic agent dispensed by the pump 100. Accordingly, it is important to provide a hermetic seal around solenoid 106. In some embodiments, the attachment of barrier 104 to bore housing 102 may be the structure most likely to compromise the hermetic seal.

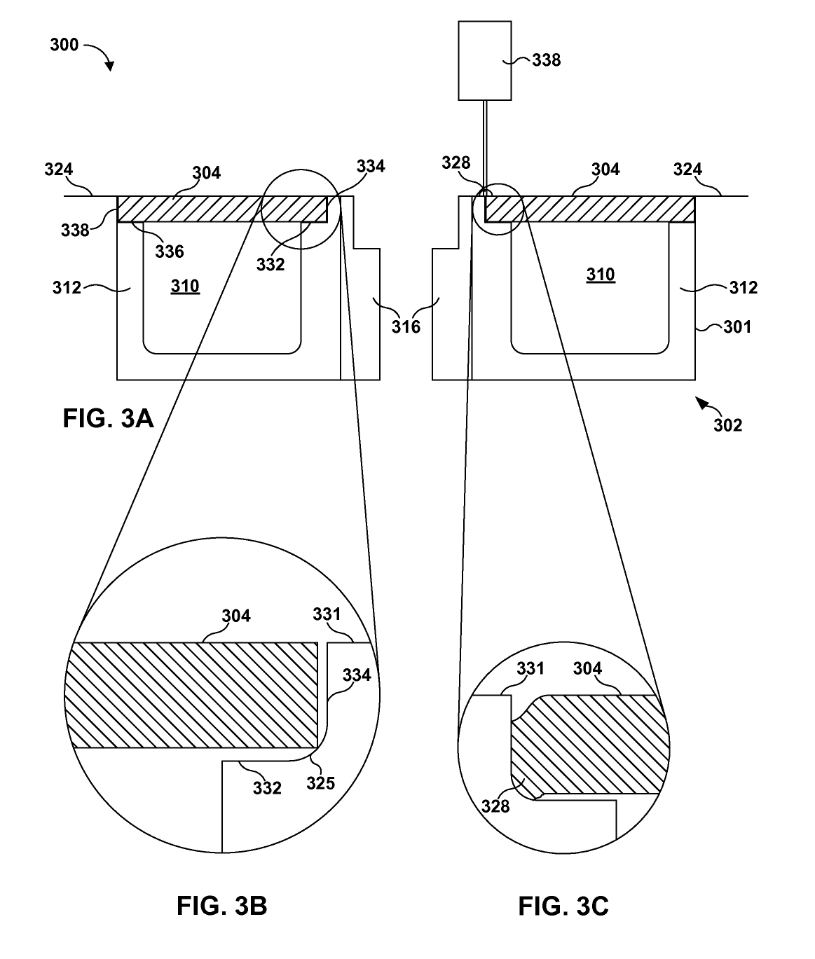

As shown in FIGS. 3A-3C, a bore housing 302 comprising a sleeve 316 and coil cup 301 may be inserted into a depression in a housing 324 of an implantable drug delivery device 300. The bore housing 302 defines a bore housing cavity 310 that barrier 304 covers. In the illustrated embodiment, horizontal surface 332 of coil cup inner wall 331 and horizontal surface 336 in bore housing outer wall 312 support barrier 304. Barrier 304 forms an intimate fit with bore housing vertical wall 334 and housing vertical wall 338.

As shown in FIG. 3B, while it may appear that barrier 304 rests directly on horizontal surface 332, barrier 304 actually contacts a fillet 325 between surface 332 and vertical wall 334. The fillet 325 may be formed because of the finite radius of curvature of the metalworking tool used to form bore housing 302 (e.g., sleeve 316). That is, the metalworking tool does not form a precise 90° angle between surface 332 and vertical wall 334 because the metalworking tool does not include a precise 90° angle.

As FIG. 3C shows, in some embodiments a welding apparatus 338 may form a butt weld 328 between barrier 304 and coil cup 301. The welding apparatus 338 may include, for example, an arc welding apparatus, an energy beam welding apparatus, or the like, and preferably includes a laser. Butt welding requires an intimate fit between the adjacent parts, in this case, a perimeter of the orifice in barrier 304 and vertical wall 334, and the outer perimeter of barrier 304 and housing vertical wall 338. Because of this, the manufacturing tolerance of each of the parts (e.g., the housing 324, bore cup 302 and barrier 304) is very tight. For example, considering only the barrier 304, a size of the outer perimeter, a geometry of the outer perimeter, a size of the orifice, a geometry of the orifice and a position of the orifice relative to the outer perimeter must be controlled to within tight tolerances. This may introduce manufacturing costs that prohibit the drug delivery device 300 from being manufactured economically, and may result in a large percentage of barriers 304 being unsuitable for use due to failing to fit properly with the vertical wall 224 and housing vertical wall 338.

Further, laser welding joins the material(s) together by melting the material(s) to form a weld without introducing any new material. This results in less material being available to make the weld 328 as the barrier 304 is made thinner, and may result in discontinuities in the weld if the melted material flows away from the welding point. These discontinuities may destroy the hermetic seal that barrier 304 is designed to provide and render the drug delivery device 300 unsuitable for implantation in a patient.

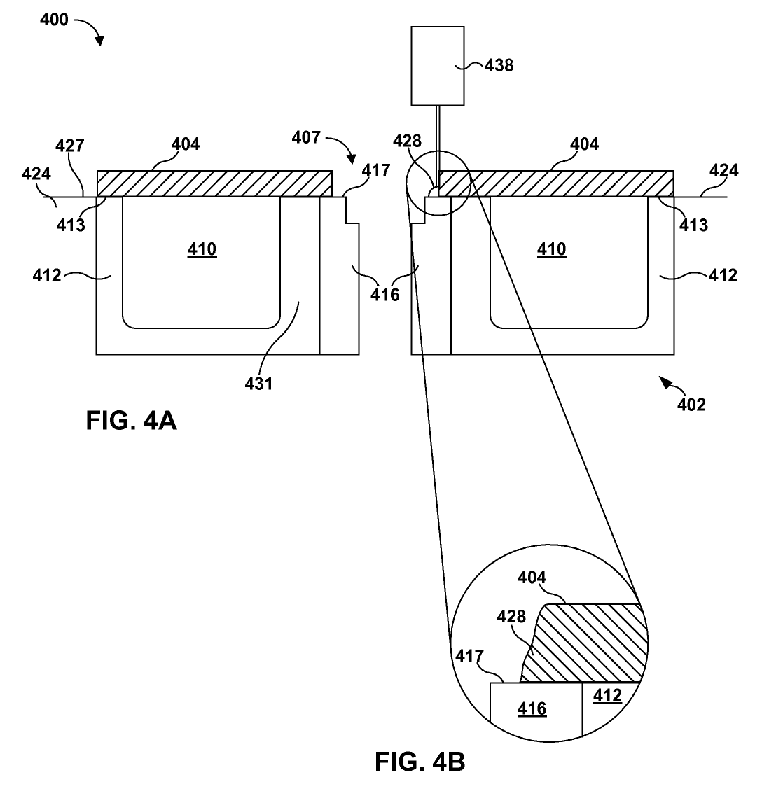

FIGS. 4A and 4B are block diagrams illustrating another embodiment of a portion of an implantable drug device, which includes a barrier that is lap welded to a bore housing and a drug device housing. As seen in FIGS. 4A and 4B, in some embodiments, the top surface 417 of the sleeve 416 is substantially coplanar with the top surface 413 of bore housing perimeter wall 412 and first surface 427 of housing 424. This allows a laser 438 to make a lap weld 428 to attach barrier 404 to sleeve 416 and housing 424 to seal bore housing cavity 410. Additionally, the use of a lap weld and the coplanar top surfaces 413 and 417 and first surface 427 increase the manufacturing tolerances of barrier 404 and reduce the complexity of bore housing 402. This lowers manufacturing complexity, and may reduce the costs associated with producing implantable drug delivery device 400.

For example, the manufacturing tolerances of the size of the outer perimeter of barrier 404, the size of the barrier orifice, and the relative location of orifice with respect to the outer perimeter of barrier 404 may be more relaxed. As one example, barrier 404 may be shifted to the left or right a certain distance in FIG. 4A and will still cover bore housing cavity 410. As another example, barrier 404 may include an orifice 407 that is not precisely centered in barrier 404, and barrier 404 may still cover bore housing cavity 410.

Additionally, the use of a lap weld alleviates the concern over a lack of material for the laser weld when the thickness of barrier 404 is reduced. For example, because the weld is no longer between two pieces of material that are aligned end-to-end, but is instead between two pieces of material, with one piece overlying the other piece, the weld effectively can utilize more material.

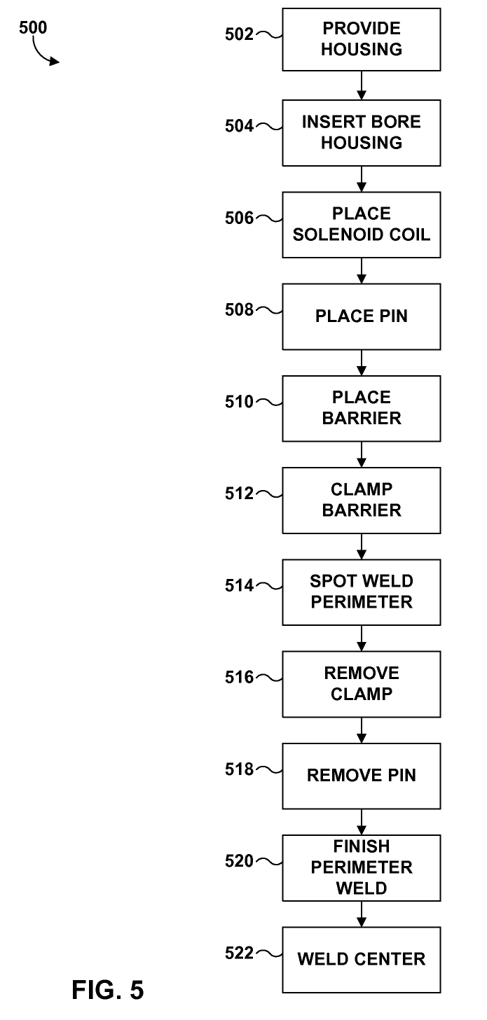

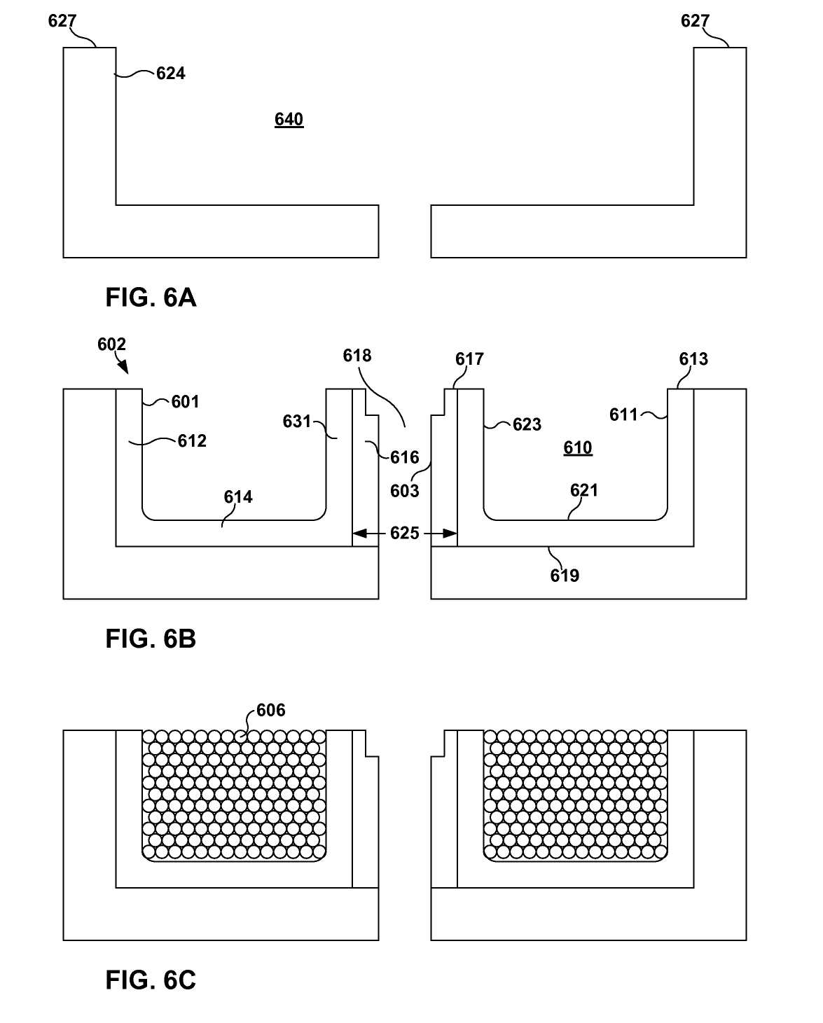

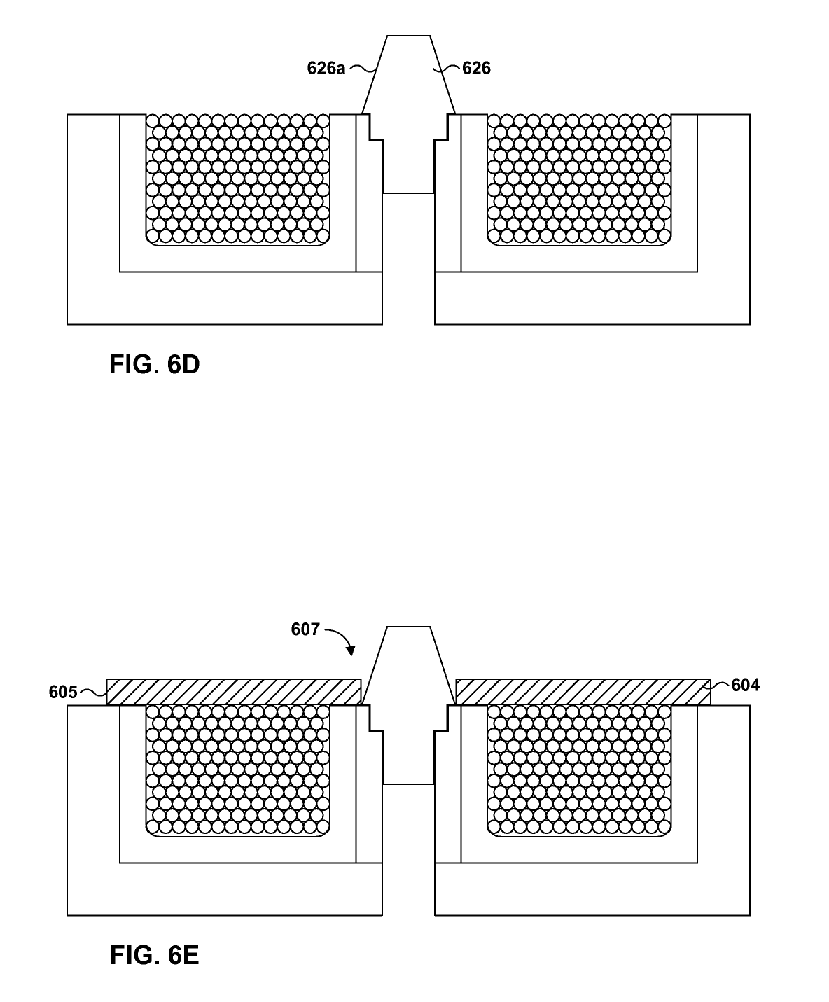

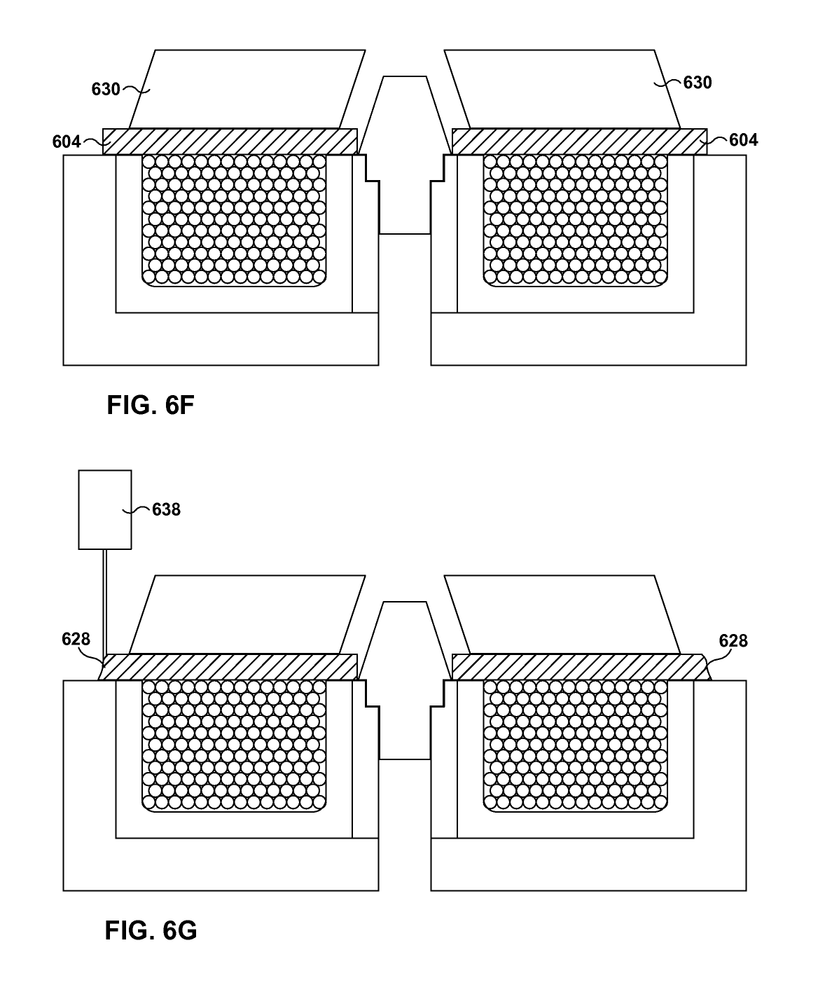

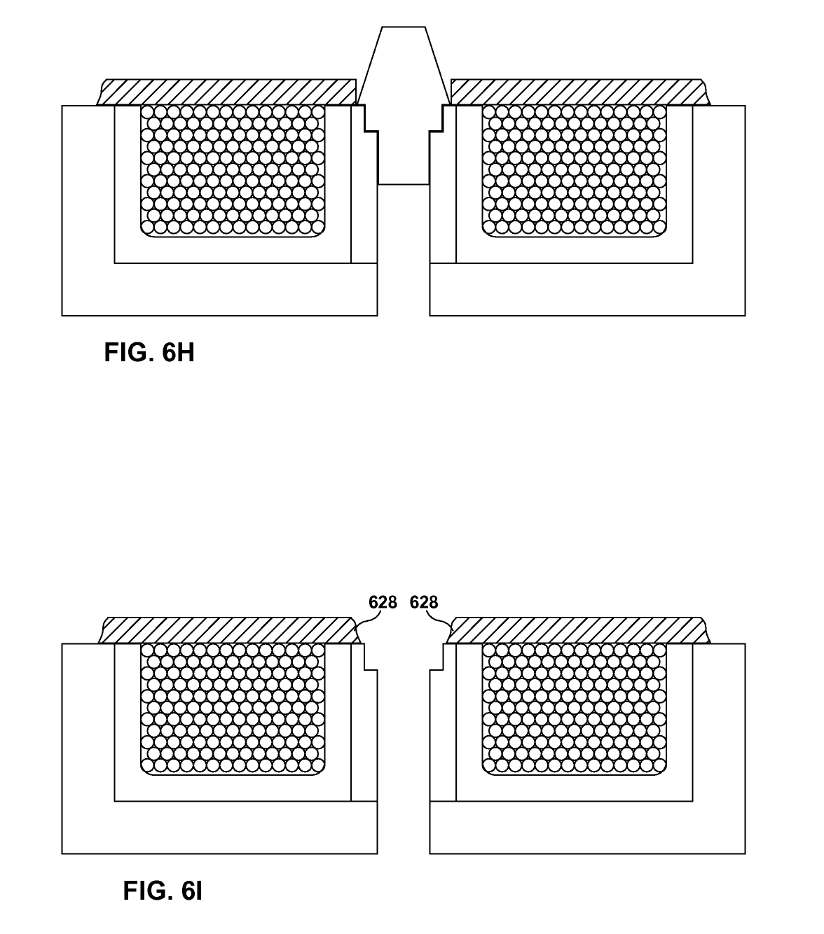

FIG. 5 is a flowchart illustrating an exemplary method 500 of forming an implantable drug delivery device including a solenoid-driven piston pump. The method 500 of FIG. 5 will be described with reference to FIGS. 6A-61 and 7. While the steps of method 500 are described as occurring in a specific order in reference to FIG. 5, the order of steps is not limited to this embodiment. For example, in other embodiments, pin 626 may be removed (520) after spot welding the perimeter (514), or after clamping the barrier (512).

First, an implantable drug delivery device housing 624 is provided (502). The housing 624 may include a first surface 627 and may define a depression 640. The housing 624 may comprise a variety of biocompatible materials, including, for example, titanium, stainless steel, and the like. While not shown in FIG. 6, the housing may contain other components of an implantable drug delivery device, such as, for example, a battery or other power source, drug reservoir, control electronics, telemetry electronics, and the like.

As shown in FIG. 6B, a bore housing 602 is inserted (504) into the depression 640. The bore housing 602 may include a coil cup 601 and a sleeve 616. Coil cup 601 includes a coil cup base plate 614 that defines a coil cup perimeter and a coil cup orifice 625. The coil cup base plate 614 includes a first major surface 621 and a second major surface 619 substantially opposite first major surface 621. The coil cup 601 may also include a coil cup perimeter wall 612 attached to the coil cup base plate 614 along the coil cup perimeter and extending substantially perpendicular to the first major surface 621 of coil cup base plate 614. Coil cup 601 may include a coil cup inner wall 331, which further defines coil cup orifice 625. Additionally, bore housing 602 may include sleeve 616 press-fit in coil cup orifice 625. A sleeve inner surface 603 defines bore housing orifice 618. A coil cup inner wall outer surface 623, first major surface 621 of coil cup base plate 614 and inner surface 611 of coil cup perimeter wall 612 define a bore housing cavity 610. Sleeve top surface 617, perimeter wall top surface 613 and first surface 627 of housing 624 may be substantially coplanar when bore housing 602 is inserted into depression 640.

Bore housing 602, including at least one of sleeve 616 and coil cup 601, may comprise a biocompatible material. For example, bore housing 602 may comprise stainless steel, titanium or the like. In some embodiments, sleeve 616 and coil cup 601 may comprise different materials. For example, in some embodiments, sleeve 616 may comprise titanium and coil cup 601 may comprise stainless steel. Bore housing 602 may include one or more additional orifices in, for example, base plate 614. The additional orifices may allow a negative pressure (e.g., vacuum) to be applied to bore housing cavity 610. This may be desired in order to secure the barrier 604 over the cavity 610 during at least part of the welding process 500.

As FIG. 6C shows, a solenoid 606 is inserted (506) in bore housing cavity 610. The solenoid coil 616 may comprise conductive material, such as, for example, copper, silver, gold, a conductive alloy, or the like. The individual coils may be insulated from each other with a thin, substantially non-conductive material. The solenoid 606 may include any number of coils, and may include any useful gauge wire. The number of coils and wire gauge may be selected based on, for example, the desired magnetic field to be produced by the solenoid 606 and the current capacity of the wire. For example, the magnetic field produced by solenoid 606 may be linearly proportional to both the number of coils in the solenoid 606 and the current passing through the coils. Thus, to produce a higher magnitude magnetic field, the number of coils in the solenoid 606 may be increased, the current carried by the coils may be increased, or both.

As shown in FIG. 6D, a pin 626 is placed (508) into the bore housing orifice 618. The pin 626 protrudes from the bore housing orifice 618 and, in some embodiments, may include a sloped section 626a, which may facilitate the placement of barrier 604 over pin 626.

Barrier 604 is placed (510) over bore housing 602, as shown in FIG. 6E. As described briefly above, barrier 604 may include a barrier orifice 607 substantially centered in barrier 604. That is, barrier orifice 607 may be substantially concentric with barrier perimeter 605. However, due to manufacturing inconsistencies or manufacturing precision limitations, barrier orifice 607 may not be precisely centered in barrier 604. Further, the shape of orifice 607 may not be precisely circular. Moreover, orifice 607 may not be precisely sized with respect to bore housing orifice 618. In any of these cases, or other manufacturing imprecisions, the substantial coplanarity of first surface 627 and top surfaces 613 and 617 may facilitate the use of an imperfect barrier 604, as described above. The pin 626 may assist in substantially centering barrier orifice 607 over bore housing orifice 618 and aligning barrier 604 to cover bore housing cavity 610. In combination with substantially coplanar first surface 627 and perimeter wall top surface 613, which allow barrier 604 to rest on top surface 613, first surface 627, or both, this allows use of barriers 604 that include manufacturing imprecisions that would have prevented their use in the drug delivery device 300 of FIGS. 3A-C.

For example, when orifice 607 is not precisely centered in barrier 604, pin 626 may align the perimeter of orifice 607 with sleeve top surface 617 so that welding of barrier 604 to top surface 617 is facilitated. In some embodiments, the location of orifice 607 with respect to orifice 618 may be more important that the location of perimeter 605 with respect to perimeter wall top surface 613. For example, in some embodiments, perimeter 605 of barrier 604 may be welded to coil cup 601, housing 624, or both. Accordingly, there may be larger tolerances for the size and placement of barrier perimeter 605 than the tolerances for the placement of barrier orifice 607.

Barrier 604 may include a biocompatible material such as, for example, titanium, stainless steel, or the like, and titanium is preferred. Barrier 604 may include a thickness of less than about 0.004 inches, and in some embodiments may include a thickness of about 0.002 inches. In other embodiments, barrier 604 may include a thickness of less than about 0.002 inches.

Once barrier 604 is placed over the bore housing 602, the barrier 604 is clamped (512) against the bore housing 602 and, in some embodiments housing 624, with clamping structure 630. For example, in some embodiments, clamping structure 630 includes a base (not shown) that receives housing 624 and a moveable portion (e.g., the portion of clamping structure 630 shown in FIG. 6F) that may be moved to retain barrier 604 in position against bore housing 602. Bore housing 602 is supported by housing 624 and, thus, the base of the clamping structure 630.

Clamping structure 630 may also assist in producing or maintaining a substantially planar barrier 604. For example, clamping structure 630 may exert sufficient force against barrier 604 to correct some non-planarity in barrier 604 and/or may prevent any non-planarity from being introduced into barrier during the welding process 500.

Once the clamping structure 630 is engaged to retain barrier 604 in position against bore housing 602, a low-pressure source, or vacuum, may optionally be engaged to further restrain barrier 604 against bore housing 602. For example, as described briefly above, bore housing 602 may include one or more additional orifice (not pictured) which may be used to fluidically couple cavity 610 to a vacuum source. The vacuum source may produce a low pressure in cavity 610, which may assist in restraining barrier 604, and may also reduce or eliminate any non-planarity in barrier 604, either alone or in conjunction with clamp 630.

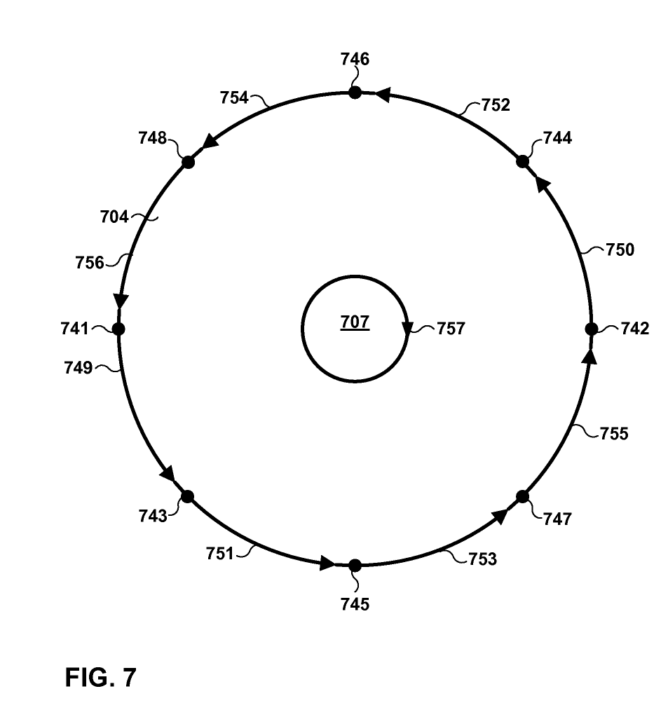

As shown in FIG. 6G, a laser 638 may spot weld (514) the perimeter 605 of barrier 604 at a plurality of locations. FIG. 7 shows further details of the spot welding. For example, the laser 638 may spot weld barrier 704 to the bore housing (e.g., coil cup 601 of bore housing 602), housing (e.g., housing 624), or both at eight discrete spot weld locations 741-747. In other embodiments, the laser 638 welds barrier 704 to the bore housing, housing, or both at more than eight spot weld locations, or less than eight spot weld locations. For example, in some embodiments, laser 638 welds barrier 704 to the bore housing, housing, or both in at least three spot weld locations.

In some embodiments, such as the embodiment shown in FIG. 7, the laser 638 completes spot welds at spot weld locations 741-748 in a sequence analogous to tightening a drum. For example, laser 638 may complete a spot weld at spot weld location 741 first, followed by a spot weld at spot weld location 742, which is located on barrier 704 substantially opposite spot weld location 741. Next, laser 638 may complete a spot weld at spot weld location 743, which is located about 45° clockwise from spot weld location 741 and substantially opposite spot weld 742 (i.e., located as far away from spot weld 743 as possible while maintaining substantially even spacing between adjacent spot weld locations around the perimeter of barrier 704), followed by a spot weld at spot weld location 744, which is substantially opposite spot weld location 743. This pattern may continue until laser 638 completes spot welds at all spot weld locations 741-748, with each weld 741-748 evenly spaced around the perimeter of barrier 704 (e.g., located about 45° from an adjacent weld 741-748). For example, laser 638 may complete a spot weld at spot weld location 745, which is located about 45° clockwise from spot weld location 743, after the spot weld at spot weld location 744, followed by a spot weld at spot weld location 746.

Once the laser 638 completes spot welds at spot weld locations 741-748, the clamp 630 may be removed (516), the pin 626 may be removed (518), and the laser 638 may weld (520) the remainder of the perimeter 603, as shown in FIG. 6H. In other embodiments, the pin 626 may be removed after spot welding the perimeter (514), or after clamping the barrier (512). As shown in FIG. 7, in some embodiments the laser 638 may begin the perimeter weld 749 at a location proximate a spot weld location 741, but a distance before location 741. In other embodiments, laser 638 may begin the perimeter weld 749 at a location proximate a spot weld location (e.g., spot weld location 741), but a distance after location 741. This may prevent the spot weld at location 741 from melting prior to a portion of perimeter weld 749 forming. The laser 638 may weld perimeter 603 in a continuous arc until the entire perimeter is welded, or may weld a plurality of arcs 749-756 until the entire perimeter is welded.

In the embodiment shown in FIG. 7, the laser 638 welds a plurality of arcs 749-756 in a pattern similar to the spot welds at spot weld locations 741-748. For example, laser 638 may weld arc 749 first. Arc 749 may begin proximate to, but before, spot weld 741. In other embodiments, arc 749 may begin proximate to, but after, spot weld 741. Arc 749 may terminate before spot weld 743. The laser 738 then may weld arc 750, which is substantially opposite arc 749 and begins proximate to, but before, spot weld 742. The laser 638 may then return to a location approximately at the end of arc 749, but overlapping arc 749, and begins to weld arc 751, which terminates proximate to, but before, spot weld 745. This pattern may continue until the entire perimeter 603 is welded. In other embodiments, the laser 638 may weld perimeter 603 in more than eight arcs, less than eight arcs, or in a continuous manner.

In the embodiments illustrated in FIG. 5, once the laser 638 completes the weld of perimeter 603, the laser 638 may weld (522) the perimeter of orifice 707 to sleeve top surface 617. The laser 638 may weld the perimeter of orifice 707 in a continuous arc 757, or may weld the perimeter of orifice 707 in a plurality of discrete arcs.

Various embodiments of the invention have been described. These and other embodiments are within the scope of the following claims.

1. A method comprising:
providing an implantable drug delivery device comprising a housing defining a depression, wherein the housing comprises a first surface;
inserting a bore housing comprising a coil cup and a sleeve in the depression, wherein the coil cup comprises:
a coil cup base plate having a first major surface and a second major surface opposite the first major surface, wherein the coil cup base plate defines a coil cup perimeter and a coil cup orifice located substantially centered in the coil cup base plate; and
a coil cup perimeter wall comprising a perimeter wall top surface, attached to the first major surface of the coil cup base plate along the coil cup perimeter and projecting substantially perpendicular to the first major surface of the coil cup base plate; and
a coil cup inner wall attached to the first major surface of the coil cup base plate along a perimeter of the coil cup orifice and projecting substantially perpendicular to the first major surface of the coil cup base plate, wherein the coil cup inner wall further defines the coil cup orifice, and wherein an outer surface of the coil cup inner wall, an inner surface of the coil cup perimeter wall and the first major surface of the coil cup base plate define a bore housing cavity, and
wherein the sleeve comprises:
a sleeve top surface and a sleeve inner surface, wherein the sleeve is press fit into the coil cup orifice, wherein the sleeve inner surface defines a bore housing orifice, and wherein when the bore housing is inserted in the depression, the first surface of the housing, the perimeter wall top surface and the sleeve top surface are substantially co-planar;
placing a solenoid in the bore housing cavity;
placing a pin in the bore housing orifice;
placing a barrier over the bore housing, wherein the barrier defines a barrier perimeter and a barrier orifice substantially centered in the barrier, wherein the pin substantially centers the barrier orifice over the bore housing orifice, and wherein the barrier substantially covers the bore housing cavity;
clamping the barrier against the bore housing with a clamping structure;
spot welding the barrier perimeter to at least one of the first surface of the housing and the perimeter wall top surface at a plurality of spot weld locations with a laser;
removing the clamping structure;
removing the pin;
welding a remaining section of the barrier perimeter to at least one of the first surface of the housing and the perimeter wall top surface with the laser; and
welding a perimeter of the barrier orifice to the sleeve top surface with the laser.

2. The method of claim 1, further comprising applying a vacuum to the cavity of the bore housing after clamping the barrier over the bore housing with a clamping structure.

3. The method of claim 1, wherein the barrier comprises Ti.

4. The method of claim 1, wherein the sleeve comprises Ti.

5. The method of claim 1, wherein the barrier comprises a thickness of less than about 0.004 in.

6. The method of claim 1, wherein the barrier comprises a thickness of about 0.002 in.

7. The method of claim 1, wherein the barrier comprises a thickness of less than about 0.002 in.

8. The method of claim 1, wherein spot welding the barrier perimeter comprises spot welding the barrier perimeter in at least three spot weld locations.

9. The method of claim 8, wherein spot welding the barrier perimeter comprises spot welding the barrier perimeter at eight spot weld locations.

10. The method of claim 8, wherein each of the at least three spot weld locations is substantially evenly spaced around the barrier perimeter.

11. The method of claim 10, wherein each subsequent spot welding location is substantially opposite an immediately previous spot welding location.

12. The method of claim 1, wherein welding a remaining section of the barrier comprises welding a remaining section of the barrier to at least one of the first surface of the housing and the perimeter wall top surface in at least one arc with the laser.

13. The method of claim 12, wherein each of the at least one arcs begins proximate to, but before or after, one of the plurality of spot weld locations.

14. An implantable drug delivery device comprising:
a housing comprising a depression and a first surface;
a bore housing inserted in the depression, wherein the bore housing comprises a coil cup and a sleeve, and wherein the coil cup comprises:
a coil cup base plate having a first major surface and a second major surface opposite the first major surface, wherein the coil cup base plate defines a coil cup perimeter and a coil cup orifice located substantially centered in the coil cup base plate; and
a coil cup perimeter wall comprising a perimeter wall top surface, attached to the first major surface of the coil cup base plate along the coil cup perimeter and projecting substantially perpendicular to the first major surface of the coil cup base plate; and
a coil cup inner wall attached to the first major surface of the coil cup base plate along a perimeter of the coil cup orifice and projecting substantially perpendicular to the first major surface of the coil cup base plate, wherein the coil cup inner wall further defines the coil cup orifice, and wherein an outer surface of the coil cup inner wall, an inner surface of the coil cup perimeter wall and the first major surface of the coil cup base plate define a bore housing cavity, and
wherein the sleeve comprises:
a sleeve top surface and a sleeve inner surface, wherein the sleeve is press fit into the coil cup orifice, wherein the sleeve inner surface defines a bore housing orifice, and wherein the first surface of the housing, the perimeter wall top surface and the sleeve top surface are substantially co-planar.

15. The implantable drug delivery device of claim 14, further comprising a barrier placed over the bore housing and laser welded to at least one of the first surface of the housing and the perimeter wall top surface, wherein the barrier defines a barrier perimeter and a barrier orifice substantially centered in the barrier, wherein the barrier orifice is substantially centered over the bore housing orifice, and wherein the barrier substantially covers the bore housing cavity.

16. The implantable drug delivery device of claim 15, wherein the barrier comprises Ti.

17. The implantable drug delivery device of claim 14, wherein the sleeve comprises Ti.

18. The implantable drug delivery device of claim 15, wherein the barrier comprises a thickness of less than about 0.004 in.

19. The implantable drug delivery device of claim 18, wherein the barrier comprises a thickness of about 0.002 in.

20. The implantable drug delivery device of claim 18, wherein the barrier comprises a thickness of less than about 0.002 in.

21. The implantable drug delivery device of claim 14, further comprising a solenoid disposed within the bore housing cavity.
